(19) United States
(12) Patent Application Publication
(10) Pub. No.: US 2007/0299422 A1
(43) Pub. Date: Dec. 27, 2007

(54) Surgical device, method for operation thereof and body-implantable device

(76) Inventors: Olle Inganas, Linkoping (SE); Edwin Jager, Linkoping (SE); Anders Selbing, Linkoping (SE)

Correspondence Address: HARNESS, DICKEY & PIERCE, P.L.C., P.O. BOX 8910, RESTON, VA, 20195, US

(21) Appl. No.: 11/797,564

(22) Filed: May 4, 2007

Related U.S. Application Data
(63) Continuation-in-part of application No. 11/467,875, filed on Aug. 28, 2006.
Application No. 11/467,875 is a continuation-in-part of application No. 10/018,985, filed on Dec. 19, 2001.

(30) Foreign Application Priority Data
Jun. 21, 1999 (SE) SE9902348-3

(51) Int. Cl. A61M 31/00

(52) U.S. Cl. 604/508; 604/57; 606/191

(57) ABSTRACT

A surgical device comprises an insertion device, adapted for insertion into a body, a tool, which is to be inserted and left in the body, and a polymer microactuator, arranged to releasably retain the tool in or on the insertion device.

### CROSS REFERENCE TO RELATED APPLICATIONS

This is a continuation-in-part of U.S. Ser. No. 11/467,875 (published as US2006/0287644A1), filed on 28 Aug. 2006, which is a continuation-in-part of U.S. Ser. No. 10/018,985, filed on 19 Dec. 2001, which is a national stage application of PCT/SE2000/001286 (published as WO00/78222A1), filed on 18 Jun. 2000, which claims priority from SE9902348-3 (now SE 519 023), filed on 21 Jun. 1999.

### TECHNICAL FIELD

The present disclosure concerns micro-surgical tools that can be delivered, grasped or caught through or by a catheter, needle, or other tube-like device. These tools or micro-structures can be used to adapt, assemble, separate, fortify, dilate, close and hold biological structures inside the body during and after surgery. The tools may be stents, valves, clips, nets, knives, scissors, dilators, clamps, tweezers etc.

The present disclosure particularly relates to a device for implanting a tool, which in particular may be an aneurysm coil, into a human or animal body. More particularly, the present disclosure relates to an implanting device, which is arranged to hold the tool during introduction into the body, and to release it at the desired position.

### BACKGROUND

The use of microstructures to assemble, fortify or dilate biological structures inside the body during and after surgery can help the surgeon in a number of ways. The operation of electrically actuated tools can help the surgeon to simultaneously position, operate manually, and observe. By positioning the tool by hand and separately operating it through external control (i.e. footswitch, voice control, other software-control) a much higher degree of precision is expected. In microsurgery, this is an especially desired advantage.

To be able to apply, beforehand or during an invasive procedure, a tool of a required size and geometry-designed for the purpose of cutting, drilling, holding, dilating, suturing, adapting or supporting-from tools that, for example, could be introduced through, placed inside or located at the end of a catheter or needle, is another desired function, requiring development of microactuators.

The application of structures in or introduced through a catheter or needle, etc is of particular interest at the application of tools, which are to be left at the site after insertion, and which have to execute their function for some limited time duration. The first example here is that of clips for surgery, sub-millimeter to millimeter structures, which would be used to hold two separated biological structures joined, for example during a healing period. Another example is that of structures for controlling the flow through blood vessels. The simplest level is that of a clip used to prevent blood flow to a biological structure downstream in the blood flow. Such a clip, or series of clips, would be mounted and left to hold a firm grip on the blood vessel and thus to prevent the flow of blood.

The third example is at a somewhat more complex level with structures built in a geometry where they could be used inside or outside tube-like structures, as so called stents to dilate a stenotic area or to internally or externally fortify or join the structure(s).

Stents are of particular interest since they are to be inserted inside the tube, then to be left there to expand a stenotic (examples: blood vessel, biliary duct) or to fortify a weak (examples: blood vessel with aneurysm, divided biliary duct) part of a tubular structure.

Arrays of fingers could be used to hold cylindrical objects, such as nerves and nerve fibers, or blood vessels. With the help of microactuators holding the structures, adjacent microstructures operating as neural sensing or activating electrodes, will enable recording signals from or activating nerves. This could be used as a synthetic neural connector, bridging a severed nerve or nerve fiber.

Elements with some temporary mechanical function could be inserted in membranes. Insertion devices of this kind could be used for mounting a hole through a membrane such as commonly used in ear surgery for pressure equilibration. Making these as microdevices will much decrease the effort to place and remove the inserted devices and to keep them in place during the desired time period.

Clips, stents, finger arrays and insertion devices, once applied, could be resorbable or permanent. They could express various degrees of stimulation of cell growth on its surfaces, various degrees of anti-thrombotic activity as well as different antibiotic activities. They can also be carriers of various biochemical or biological components.

Electroactive polymers (EAP) are a comparatively novel class of materials that have electrically controlled properties. An overview on electroactive polymers can be found in “Electroactive Polymers (EAP) Actuators as Artificial Muscles—Reality, Potential, and Challenges” 2nd ed. Y. Bar-Cohen (ed.) ISBN 0-8194-5297-1.

One class of EAPs is conducting polymers. These are polymers with a backbone of alternating single and double bond. These materials are semiconductors and their conductivity can be altered from isolating to conducting with conductivities approaching those of metals. Polypyrrole (PPy) is one conducting polymer and will throughout the present disclosure be taken as a non-limiting example of such materials.

Polypyrrole can be electrochemically or chemically synthesised from a solution of pyrrole monomer and a salt as is known to those skilled in the art. After synthesis PPy is in its oxidised, or also called doped, state. The polymer is doped with an anion A−.

PPy can be electrochemically oxidised and reduced by applying the appropriate potential to the material. This oxidation and reduction is accompanied with the transport of ions and solvents into and out of the conductive polymer. This redox reaction changes the properties of polypyrrole, such as the conductivity, colour, and volume.

Two different schemes of redox are possible. If PPy is doped with a large, immobile anion A− scheme 1 occurs, which schematically can be written as:

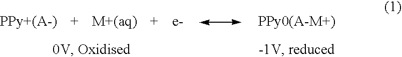

When PPy is reduced to its neutral state cations M+ including their hydration shell and solvent are inserted into the material and the material swells. When PPy is oxidised again the opposite reaction occurs, M+ cations (including hydration shell and solvent) leave the material and it decreases its volume.

If on the other hand PPy is doped with small, mobile anions a−, scheme 2 occurs:

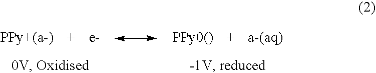

In this case the opposite behaviour of scheme 1 occurs. In the reduced state the anions leave the material and it shrinks. The oxidised state is now the expanded state and the reduced state the contracted. Non limiting example of ions A− is dodecylbenzene sulfonate (DBS−), of a− perchlorate (ClO4−), and of M+ sodium (Na+) or lithium (Li+) This volume change for instance can be used to build actuators (See Q. Pei and O. Inganäs, “Conjugated polymers and the bending cantilever method: electrical muscles and smart devices”, Advanced materials, 1992, 4(4), p. 277-278. and Jager et al., “Microfabricating Conjugated Polymer Actuators”, Science 2000 290: 1540-1545).

This redox reaction needs to be driven in an electrochemical cell that comprises a working electrode (i.e. the conducting polymer) and a counter electrode, preferably a reference electrode, and an electrolyte.

The electrolyte is preferably an aqueous salt solution, but can be a solid polymer electrolyte, gels, non-aqueous solvents, ionic liquids as is known to those skilled in the art, but even biologically relevant environments such as blood (plasma), cell culture media, or other physiological media, etc. can be used.

However, little is known about how to design and position EAP actuators for optimal operation in connection with implantation devices.

Furthermore, little is known about how to design and position an EAP actuator-based release mechanisms to achieve sufficient and reliable clamping and/or release effect.

A brain aneurysm, is an abnormal bulging outward of one of the arteries in the brain. A brain aneurysm can also be called a cerebral or intracranial aneurysm.

Cerebral aneurysms are treated using endovascular techniques. In the procedure, a catheter is guided from the femoral artery, up through the aorta, and into the cerebral vasculature either via the carotid or vertebral artery until it reaches the aneurysm. Tiny (platinum) coils are normally threaded through the catheter until the aneurysm is packed with enough coils to prevent blood flowing into it and also preventing rupture. This process is called embolization. Aneurysms can also appear in blood vessels in other parts of the body (outside the brain) and similar embolization techniques can be applied here.

Present methods take a long time to release the coil from the “push rod” (delivery/positioning unit), up to 30 s. Taking into consideration that many coils are to be inserted into the aneurysm, one understands that release time is a critical issue. There is a need for alternative methods, or for methods that can reduce the release time.

Delivery methods available today includes incorporation of a polyethylene fiber between a delivery/positioning unit (e.g. guidewire) and the coil to be released. Deployment of the microcoil occurs when a resistive heater is activated at the tip of the delivery unit, shearing the polyethylene fiber. This technique optimally deploys a coil within five seconds. Delivery using pressure: A micro-fluidic detachment mechanism is used to generate localized pressure within a detachment coupler zone. The pressure gently releases the coil from the delivery pusher.

Another delivery method includes electrolytical detachment of coils, meaning that the coil is attached to the delivery/positioning device and is then separated therefrom electrolytically.

The combination of microactuators and catheters is not well documented in the literature.

A patent search reveals a few examples but none that describes the use of microactuators as tools housed inside a catheter; several examples of microactuators use to position a catheter are to be found in U.S. Pat. No. 5,771,902, U.S. Pat. No. 5,819,749, W09837816A1, W09739688A2, W09739674A1 and U.S. Pat. No. 5,855,565.

Several mechanisms are suggested for the microactuators in these applications, found among shape memory alloys (including polymeric materials) and piezoelectric materials. The use of conjugated polymers in micromuscles/microactuators is not documented for catheter tools.

Hence, there is no previous disclosure of the use of microactuators based on conjugated polymers being electrically operated and mounted in or on a catheter or needle, to be positioned with the help of the catheter, and then activating the microactuator structures carried on the needle.

The microfabrication of such microactuators renders possible a number of geometries from 10 μm and larger, difficult to produce by mechanical production techniques. They may be produced by use of the method presented in the documents referred to above and then mounted in or on the needle or catheter, or they might be produced by novel manufacturing methods.

In view of the above, there is a need for an improved or alternative device for implanting a tool in the human or animal body.

### SUMMARY

It is a general objective of the present disclosure to provide a device which eliminates or at least alleviates some or all of the disadvantages of the prior art.

A particular objective is to provide a device which enables accurate, reliable and fast delivery of a body-implantable tool. Such tools may be intended for temporary or permanent implantation into the body.

The above referenced objectives are at least partially achieved by a device according to the appended independent claim.

Embodiments are set forth in the appended dependent claims, in the following description and drawings.

According to a first aspect, there is provided a surgical device, comprising an insertion device, adapted for insertion into a body, a tool, which is to be inserted and left in the body, and a polymer microactuator, arranged to releasably retain the tool in or on the insertion device.

The polymer microactuator may comprise an electroactive polymer material, which changes volume upon actuation.

The tool may be releasably retainable inside the insertion device.

The insertion device may comprise a catheter or a cannula.

A carrier device may be insertable into the insertion device.

The tool may be releasably retainable on the carrier device.

The carrier device may comprise a needle.

According to a second aspect, there is provided a method for operating a surgical device, comprising providing an insertion device, adapted for insertion into a body, providing a tool, which is to be inserted and left in the body, and which is releasably attached to the insertion device, and actuating a polymer microactuator, so as to release the tool from the insertion device.

According to a third aspect, there is provided a body-implantable device, comprising first and second passive device portions, and an attachment device for releasably attaching said first and second device portions to each other, wherein said attachment device comprises a polymer microactuator.

By “passive” is meant that the portions are substantially unaffected by such actuation as is necessary for the polymer microactuator to operate.

Such a device may be used in situations, where a temporary constriction, such as a limited diameter, is desirable, followed by a controlled increase, which may be instantaneous or stepwise.

The first and second device portions may be formed in one piece.

The device may further comprise a retaining portion, arranged to retain said first and second portions subsequent to said actuation of the polymer microactuator.

The retaining portion may be arranged to, subsequent to said actuation, retain said first and second portions at a larger distance from each other than prior to said actuation.

The first and second parts may be arranged to form an encircling device, the circumference of which being larger subsequent to said actuation, than prior to said actuation.

The necessary elements to accomplish these functions may be the electrochemically activated micromuscles, built by micromachining thin metal and polymer layers (Elisabeth Smela, Olle Inganäs and Ingemar Lundström: “Controlled Folding of Micron-size Structures”, Science 268 (1995) pp. 1735-1738) or only polymer layers. These actuators can be produced in sizes from micrometers to centimeters, and operate well in biological fluids such as blood plasma, blood, buffer and urine. They are therefore suitable tools for micro invasive surgery inside the body.

The versatility of construction and the speed of response, as well as the force of these actuators render them as one of the best types of microactuators for use inside the body. An international patent covers one route of fabrication of such devices (A Elisabeth Smela, Olle Inganäs and Ingemar Lundström: “Methods for the fabrication of micromachined structures and micromachined structures manufactured using such methods”, Swedish patent application number SE 9500849-6, Mar. 10, 1995 in succession also a PCT and international patent).

The production of individually actuated tool arrays render little difficulty beyond that of producing the individual tool; we have to see that electrical contacts are supplied to actuate each microactuator separately. This can be done by wiring the single microactuator, to be used as the working electrode; the catheter is then used as the counterelectrode, and will be able to supply all the charge that we ever need to actuate all those microactuators. As wires may easily be produced in width down to 10 μm with photolithography or with soft lithography, we will be able to put at least 50 microactuators along the tool array located in/on a needle of 1 mm width, with the simple philosophy of putting down parallel conductor wires. Should we need more, more elaborate addressing schemes might be needed.

Should a necessity for three electrode systems be found in any of the applications, microfabricated reference electrodes or macrosize reference electrodes carried on the catheter housing offers a solution for this problem.

Should the tool arrays be collectively addressed, and the tool array may be designed to set free the outermost tool, which as illustrated may be a clip, on actuation of all the clips, we will need a mechanism of confining the movements of all but the outermost tool. This may be done by assembling the tool array into a cylindrical housing, preferably the catheter, prior to insertion in the body. The cylindrical housing is now confining the motion of microactuators, which search in vain to expand the strong metal casing on operation. When the outermost clip 1 is actuated, the tool is opened; likewise is the next-to-the outermost tool 4 partially free to move as it is protruding outside the cylindrical housing. Therefore the partial opening of tool 4 sets tool 1 free, as well as opens it up for subsequent spontaneous closing on the site where the tool is to be positioned.

### BRIEF DESCRIPTION OF THE DRAWINGS

All figures are schematic and not to scale, and vertically dimensions are exaggerated. Thickness ratios between the layers are not as illustrated in these figures.

FIGS. 1a-1c schematically illustrate a surgical device, comprising an insertion device, adapted for insertion into a body, a tool, which is to be inserted and left in the body, and a polymer microactuator, arranged to releasably retain the tool in the insertion device.

FIG. 2 schematically illustrates tubular tweezers, tweezers and knifes, based on microactuators.

FIGS. 3a-3b schematically illustrate a neural connector.

FIGS. 4a-4b schematically illustrate an insertion device, for making a temporally permanent hole through a membrane.

FIGS. 5a-5b schematically illustrate a stent device.

FIG. 6 schematically illustrates introduction of an aneurysm coil into an aneurysm formed in a blood vessel wall.

FIGS. 7a-7b schematically illustrate detachment of a tool (second part) from an insertion device (first part).

FIGS. 8a-8d schematically illustrate different detachment setups.

FIG. 9 schematically illustrates an array of insertable devices.

FIGS. 10a-10e schematically illustrate positions of a detachment part relative to the first and second parts.

FIGS. 11a-11j schematically illustrate different embodiments of outwardly acting detachment parts, each being illustrated in connected and disconnected state.

FIGS. 12a-12j schematically illustrate different embodiments of inwardly acting detachment parts, some of which being illustrated in connected and disconnected state.

FIGS. 13a-13e schematically illustrate different embodiments of axially acting detachment parts, each being illustrated in connected and disconnected state.

FIGS. 14a-14c schematically illustrate different embodiments of detachment parts acting in a deforming and/or rupturing manner, each being illustrated in connected disconnecting and disconnected state.

FIGS. 15a-15c schematically illustrate different embodiments of detachment parts making use of material properties, each being illustrated in connected and disconnected state.

FIGS. 16a-16b schematically illustrate different embodiments of detachment parts integrated with the second part.

FIG. 17 schematically illustrates an embodiment of a detachment part arranged on a third part.

FIGS. 18a-18b schematically illustrate bushing designs for releasing the second part.

FIGS. 19a-19b schematically illustrate an implant using separable parts.

FIGS. 20a-20b schematically illustrate yet another release mechanism, in connected and disconnected states.

FIGS. 21a-21b schematically illustrate different array release mechanisms.

FIGS. 22a-22f schematically illustrate different actuator configurations which may be used to provide a polymer microactuator.

FIGS. 23a-23h schematically illustrate different release configurations of a surgical device.

FIGS. 24a-24b schematically illustrate yet another design principle for a surgical device.

### DESCRIPTION OF EMBODIMENTS

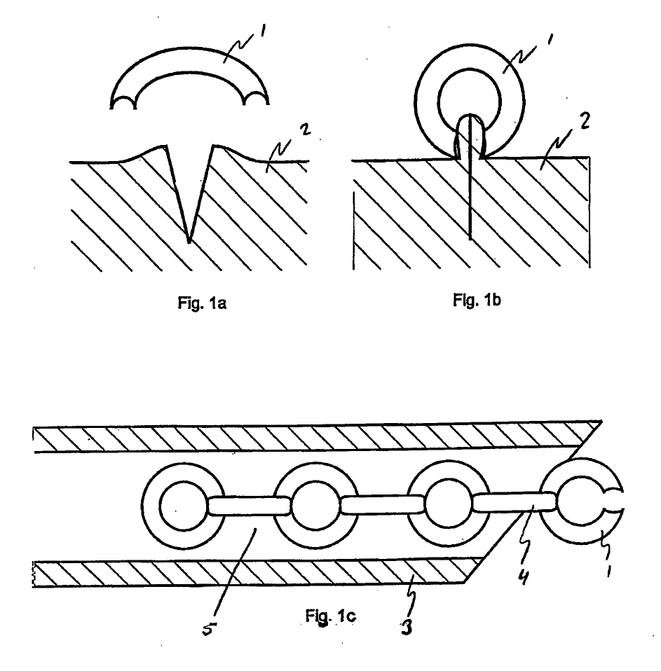

FIGS. 1a-1c schematically illustrate a surgical device, comprising an insertion device 3, adapted for insertion into a body, a tool 1, 4, which is to be inserted and left in the body, and a polymer microactuator, arranged to releasably retain the tool in the insertion device. In the example of FIG. 1c, the tools 1, 4 are clips which are mounted in sequence, and confined by a cylindrical housing. When pushed outside the end of the cylindrical housing, the outermost clip 1, opens up. Actuation, or deactuation of the clip closes it to join the open structure 2. The outermost clip 1 is then set free by the simultaneous operation of the second-to-outermost clip 4, so as to be left at the structure 2, holding the structures together.

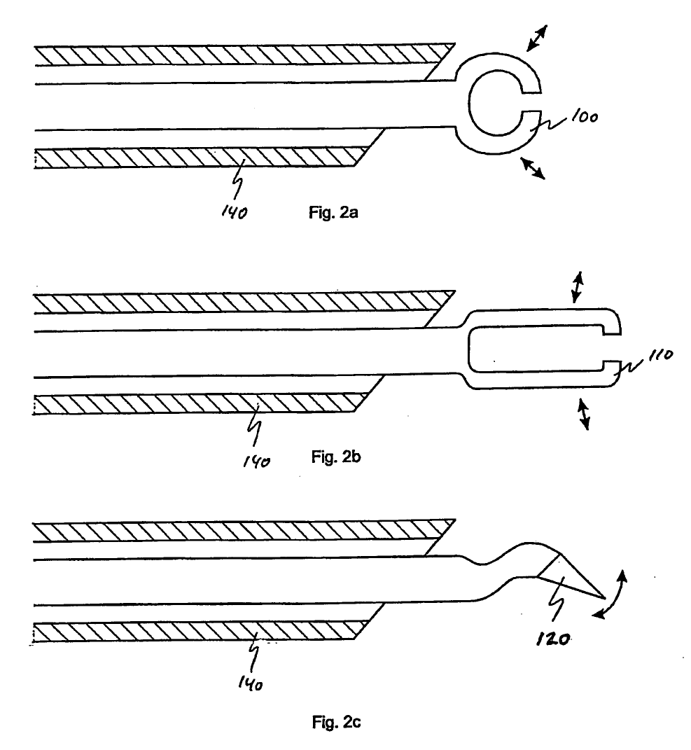

FIG. 2a-2c schematically illustrate tubular tweezers 100, tweezers 110 and knifes 120, based on microactuators. The indicated movement is driven by microactuators properly mounted and designed.

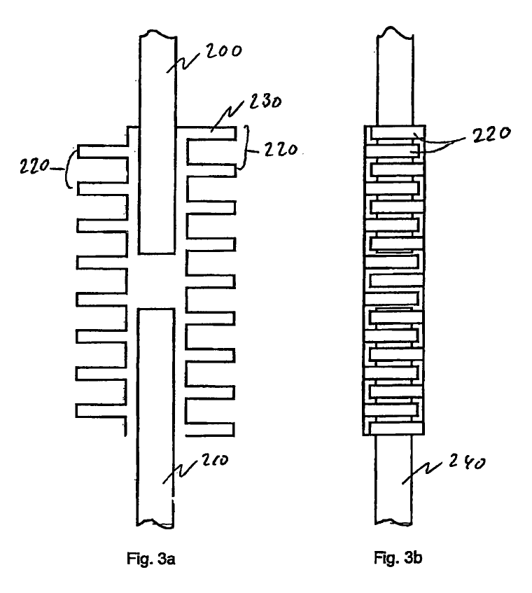

FIGS. 3a-3b schematically illustrate a neural connector 230, where a number of small fingers 220, 230 coil around a cylindrical nerve 240 to make a tight hold the nerve. Two separate nerves are here joined with the help of a common neural connector, which would be desired for accomplishing regrowth of the nerves. In addition, small electrodes can be fashioned along with the microfingers, and be used to sense or excite nerve signals.

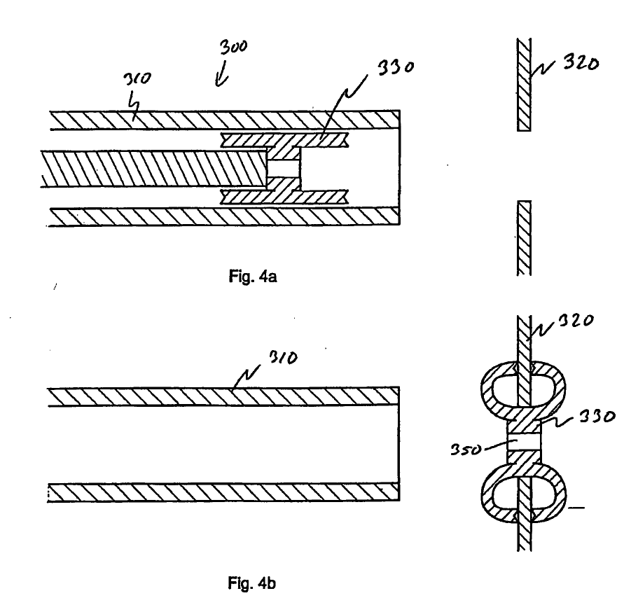

FIGS. 4a-4c schematically illustrate a device 330, for making a temporally permanent hole 350 through a membrane 320. The device 330 is housed in a catheter/cannula/needle 310 and is inserted through the membrane so as to make the device form a hole through the membrane.

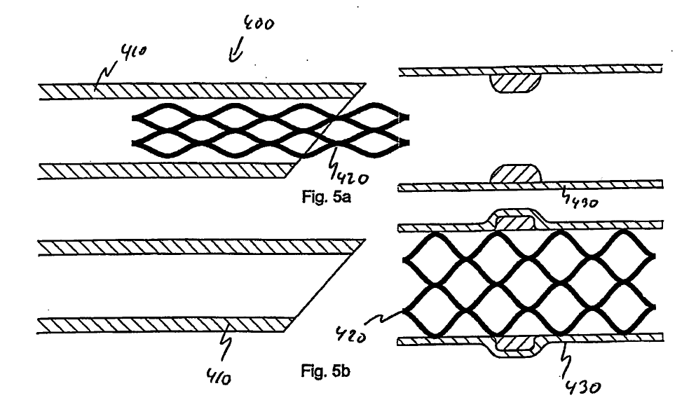

FIGS. 5a-5b schematically illustrate a stent device 420, which is insertable through a cannula or catheter 410 into a blood vessel 430.

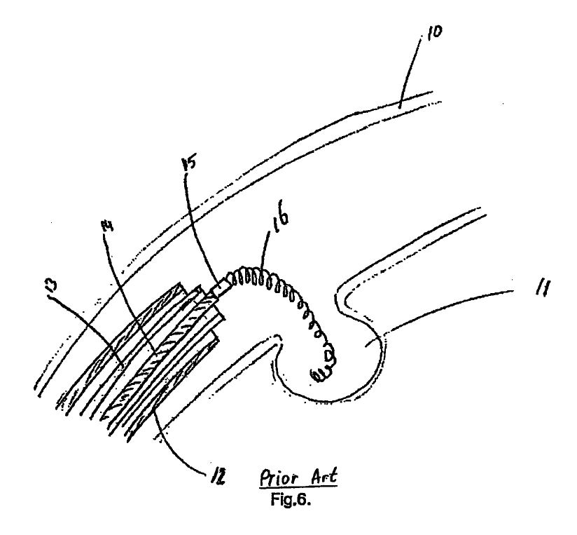

FIG. 6 shows a part of a blood vessel 10 that has an aneurysm 11. As mentioned previously one or more coil(s) 16 is inserted into the aneurysm. Typically during such a surgical procedure, first a guide catheter 12 is positioned near the aneurysm. Through the guide catheter a coil delivery/positioning device is inserted. The coil delivery device comprises a catheter/protection tube 13, a push rod (delivery/positioning unit) 14 onto which the coil 16 is mounted. A detachment part 15 is positioned between the rod 14 and coil 15 in order to release the coil using electrolytic or other methods as mentioned previously.

The remainder of the present disclosure will focus on a medical device that is used to release (or capture) a second part by using an EAP actuated releasing (or capturing) mechanism. A non-limiting example of such medical devices is the embolic coil delivery system of FIG. 6, where the detachment part 15 is actuated/triggered by EAP.

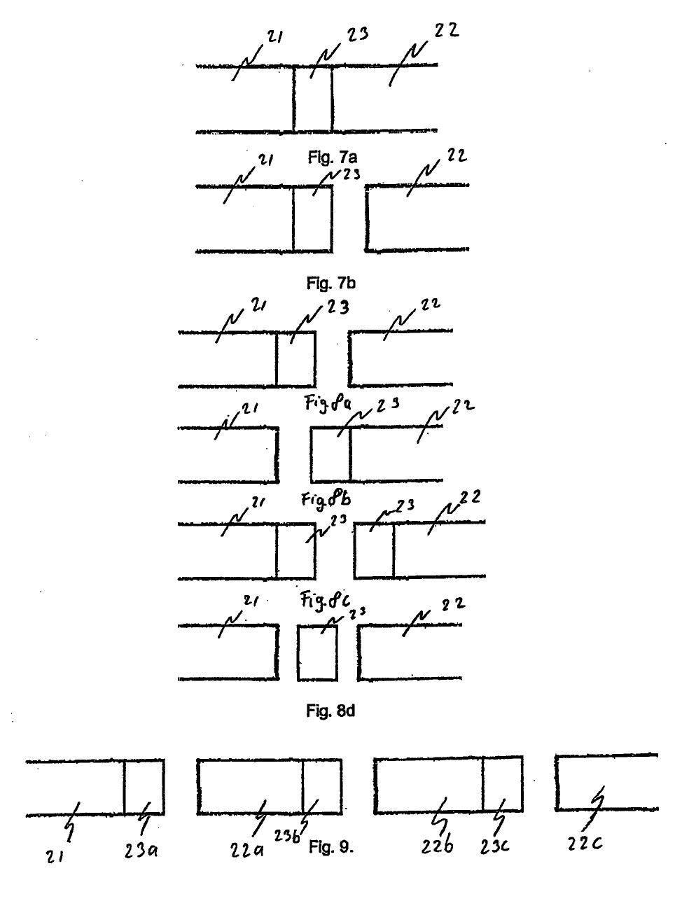

FIG. 7 illustrates the concept in its essence. The medical device 20 comprises three parts, a first part 21, and releasable or capturable second part 22, and a detachment part 23 which may comprise an EAP actuator/material, such as an electroactive material that changes volume upon actuation. To illustrate, the first part 21 may be the proximal side of a medical device or it may be the delivery tool of a medical device. The second part 22 may be the distal side of a medical device or it may be the implant to be inserted into the body. Taking FIG. 6 as an example, the first part 21 may be the push rod 14 and the second part 22 may be the embolic coil 16, and the detachment part 23 may be the detachment part 15. Further examples of part 21 may be catheters, endoscopes, guidewires, sheaths, stylets, trocars, leads, implants etc. Further examples of part 22 may be stents, grafts, clips, clamps, filters, baskets or snares for retrieval of objects, patches, embolization devices and embolization materials such as Bead Block™ micropsheres, or smart structures (MEMS, Biosensors, etc).

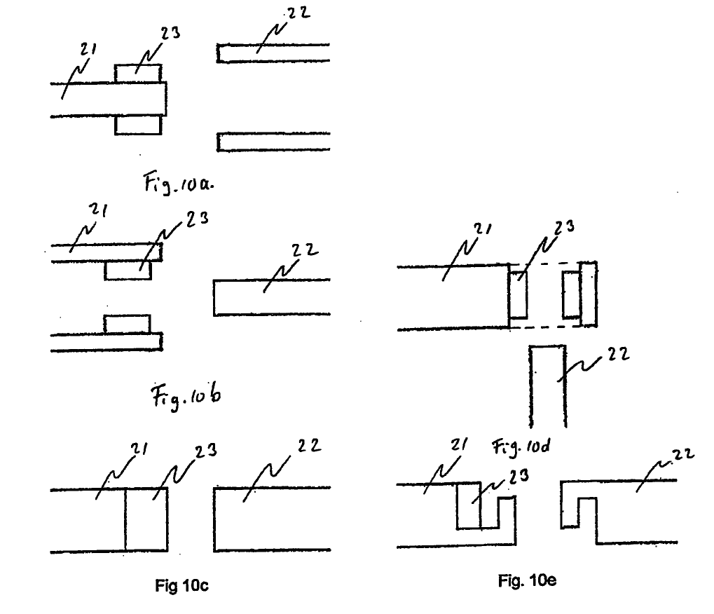

FIG. 8 illustrates different embodiments of the medical device 20 after detachment of the second part 22. The EAP actuated detachment part may be arranged on the first part 21 (FIG. 8a) or the second part 22 (FIG. 8b) or be (partly) attached on both parts 21 and 22 (FIG. 8c). In addition, the EAP actuated detachment part 23 may be a separate third part, or it may be attached to a third part (FIG. 8d).

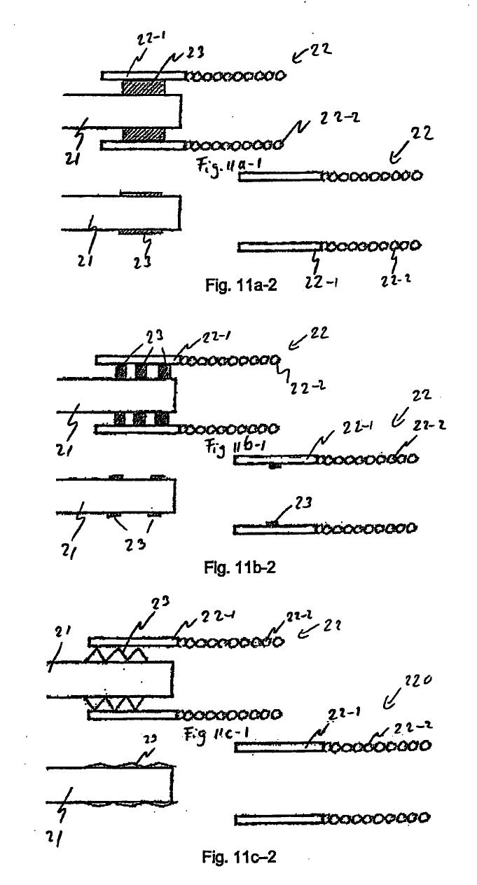

FIG. 9 illustrates an array of tools/implants/parts, where multiple tools/implants/parts may be released from the same medical device, similarly to what was illustrated with respect to FIG. 1. The part 22a is attached to the delivery device by detachment part 23a. Part 22a further comprises a detachment part 23b to which the separately releasable part 22b is attached. Releasable part 22c is in its turn attached to part 22b by detachment part 23c. The releasable parts can be individually released by first activating detachment part 23c, then 23b and last 23a. In this example the parts 22a through 22c are serially connected to each other. Likewise they may be all directly coupled to part 21.

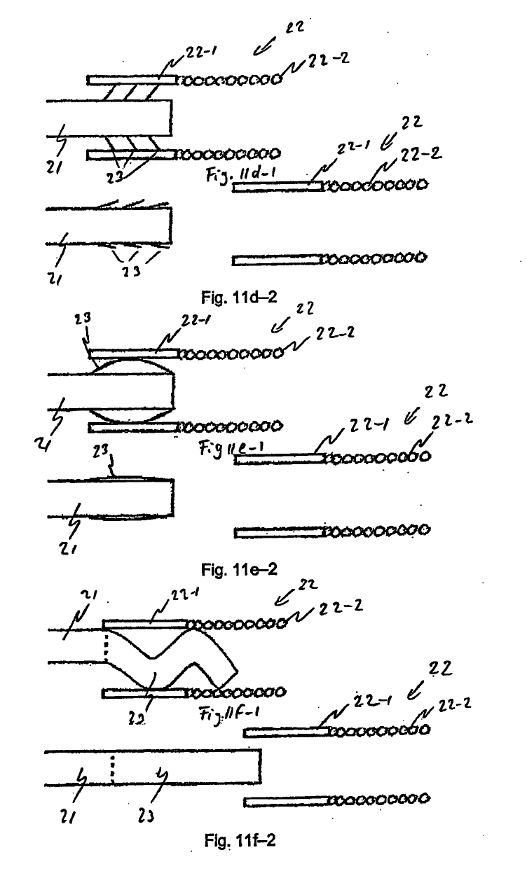

FIG. 10 illustrates different embodiments of the position of the detachment part. In the figure, the detachment part 23 is illustrated to be positioned on the first part 21, however it may be positioned on any part as illustrated in FIGS. 8 and 9. In FIG. 10a the detachment part is illustrated to be positioned on the outside of the part 21, so that it grabs/holds the second part 22 from the inside.

In FIG. 10b, the detachment part is illustrated to be positioned on the inside of the part 21, so that it grabs/holds the second part 22 from the outside.

In FIG. 10c the two parts 21 and 22 are attached end-to-end or head-to-tail.

FIG. 10d is a variant of FIG. 10b, and illustrates that the detachment part may be positioned at a different angle. In this particular example, it is positioned at 90 degrees, i.e. perpendicular to the cross section (of the FIG. 7b).

FIG. 10e illustrates, yet another embodiment of the invention. Part 22 is partly “inserted” into part 21, as compared to FIG. 10c.

In the following, embodiments of an implantation device will be described, embolic coils as a specific tool example. The implantation device comprises a first part, which may be an insertion device 21, a second part 22, which may be a tool, such as the embolic coil, and a detachment/grasping part 23, which comprises a polymer microactuator. The tool 22 may, in some embodiments, comprise an attachment portion 22-1 and a active tool portion 22-2.

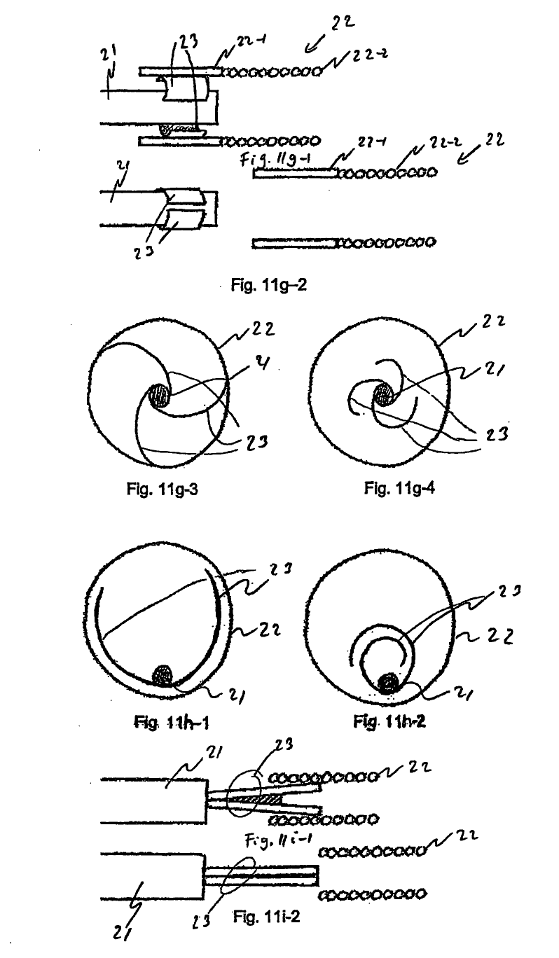

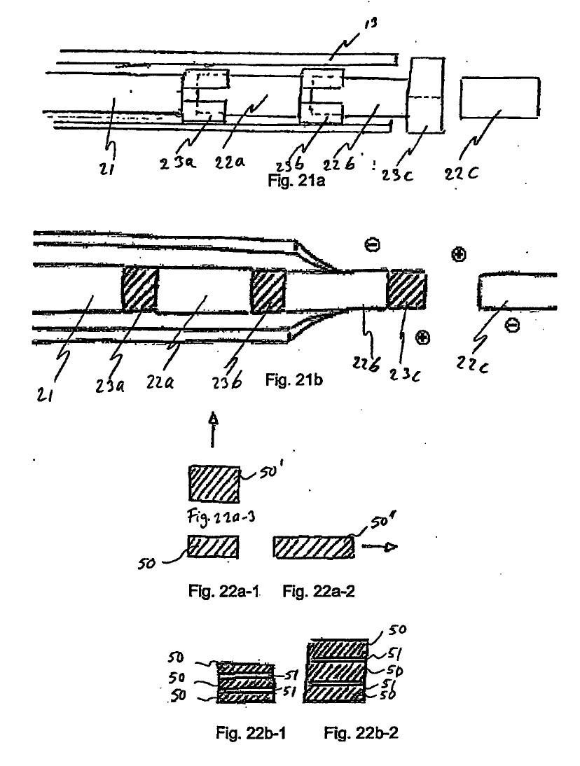

Now turning to FIG. 11, there are illustrated different embodiments of the inwards grip, with the detachment part positioned on the first part 21 i.e. a combination of FIG. 8a and FIG. 10a. FIG. 11a illustrates an EAP detachment part comprising an EAP actuator, the volume (or outer diameter) of which decreases upon activation of the EAP actuator. Such volume changing actuators are illustrated in FIGS. 22a, 22b, 22d, 22e. In the expanded phase, the part 23 holds the second part 22 on the inside and when activating, the part 23 shrinks and the part 22 is released.

FIG. 11b illustrates a variant of FIG. 11a, now having two detachment parts 23 on part 21 and one detachment part on part 22. The different detachment parts may be actuated simultaneously.

FIG. 11c illustrates an embodiment where the detachment part 23 is shaped as “bellows”, i.e. a mechanical solutions where a linear volume change in the EAP material is transformed into a perpendicular change of the actuator dimensions. (FIGS. 22d, 22e)

FIG. 11d illustrates an embodiment where the detachment part 23 is shaped as pins, spikes or benders that can fold inwards in order to release the part 22. (FIGS. 22c, 22f)

FIG. 11e illustrates an embodiment where the detachment part 23 is shaped as a balloon (which may be inflatable) or buckling membrane (FIGS. 22e).

FIG. 11f illustrates an embodiment where the detachment part 23 is shaped as bending/alternating/corkscrew like shape. Activating the part 23 straightens it and thus releasing part 22. Part 23 may be shaped as EAP controllable steerable guidewire (see US2003/0236445A1), bellows, bilayer coil, undulator (FIGS. 11c-1, 11c-2, 22e, 22f).

FIGS. 11g-11i illustrate an embodiment where the detachment part 23 is shaped as folding wings that grab the part 22 from the inside.

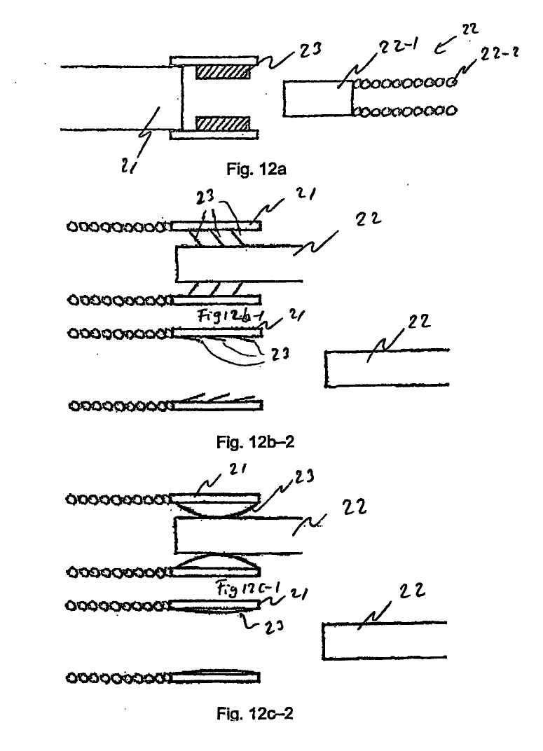

FIG. 11j illustrates a “tweezer” kind of detachment mechanism. Two tweezer arms hold the releasable part from the inside. In this case a volume changing EAP actuator is used to close the tweezer arms, but any other tweezer-like mechanism may be used (see for instance FIGS. 12g, 12h, 12i and 22c, 22f)

As the one skilled in the art realises, the release mechanisms of FIG. 11a-11c may also be applied as to generate an inwardly acting grips. A few of these are illustrated in FIG. 12. That is, a combination of FIGS. 8a and 10b.

FIG. 12a illustrates an EAP detachment part that comprises an actuator which volume decreases, or inner diameter of the gap between actuator parts expands upon activation of the EAP material. Hence, this is a variant of FIG. 11a.

FIG. 12b illustrates an embodiment where the detachment part 23 is shaped as pins, spikes, or benders that can fold outwards in order to release the part 22. Hence, this is a variant of FIG. 11d.

FIG. 12c illustrates an embodiment where the detachment part 23 is shaped as a balloon or buckling membrane (c.f. FIG. 11e).

In FIG. 12d is illustrated a pair of circumferentially enclosing benders that grab the part 22. Activating the EAP material, opens the benders like a pair of tweezers, releasing part 22. Hence, this is a variant of FIG. 11h or 11i.

The detachment part 23 as shown in FIG. 12e comprises two EAP buckling actuators (i.e. a clamped bender). Activating the EAP buckling/balloon actuators, makes the actuators buckle outwards, releasing the second part 22.

FIGS. 12f show the same embodiment at different cross sections. Activating the EAP detachment portion opens it up and thus releasing part 22 (as illustrated in 17d).

“Tweezer”-type detachment mechanisms, where two tweezer arms grab the releasable part 22 from the outside are illustrated in FIGS. 12g through 12i.

The tweezer arms may be opened using a volume expanding EAP actuator (12g and 12i) or using a pair of benders a tweezers (12i). However any other tweezer-like mechanism may be used (see for instance FIGS. 22).

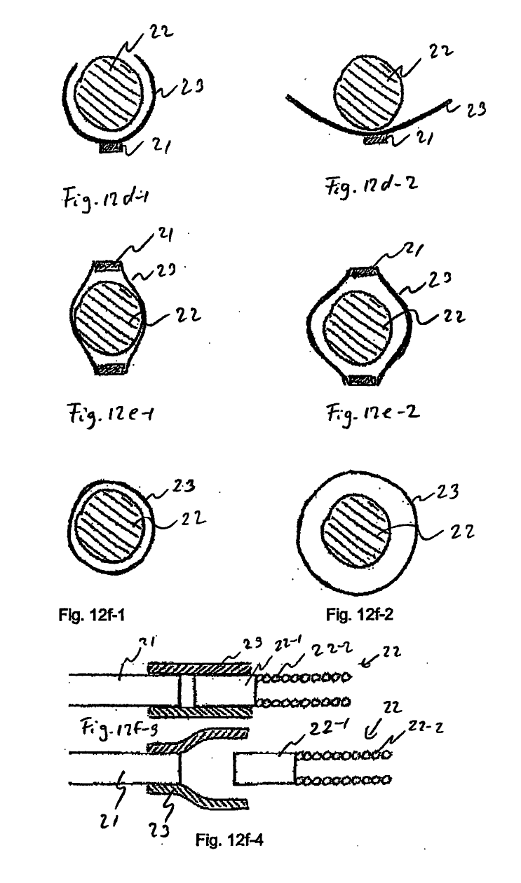

Head to tail configurations (FIG. 8a and FIG. 10c) are illustrated in FIG. 13a-13e. They may for instance use the volume, buckling, or bender EAP actuator configurations (see FIGS. 22a, 22b, 22c, 22e, 22f for details on each mechanism).

In FIG. 13a, an EAP portion having its major expansion direction in the longitudinal direction of the device is illustrated.

In FIG. 13b, a buckling membrane, arranged to buckle in the longitudinal direction of the device is illustrated.

In FIG. 13c, a bending EAP actuator is arranged to provide a bending motion, which will push the second part 22 axially out of the grip.

The detachment part 23 of FIG. 13d uses the volume configuration to “squeeze out” the part 22. In such arrangements, it may be necessary to provide interacting conical surfaces between the EAP material and the portion of the second part which interacts with the EAP material. Another option may be to actuate the EAP material from the bottom of the axial recess and outwardly.

The embodiments of FIGS. 13a-13d may also be used in a situation where the part on which the microactuator is arranged is enclosed by the other part. In such embodiments, the actuator may be arranged on the outside of its associated part, to force the other part off.

The detachment part 23 of FIG. 13e shrinks/shortens/retracts in the linear/extended direction thus releasing the part 22.

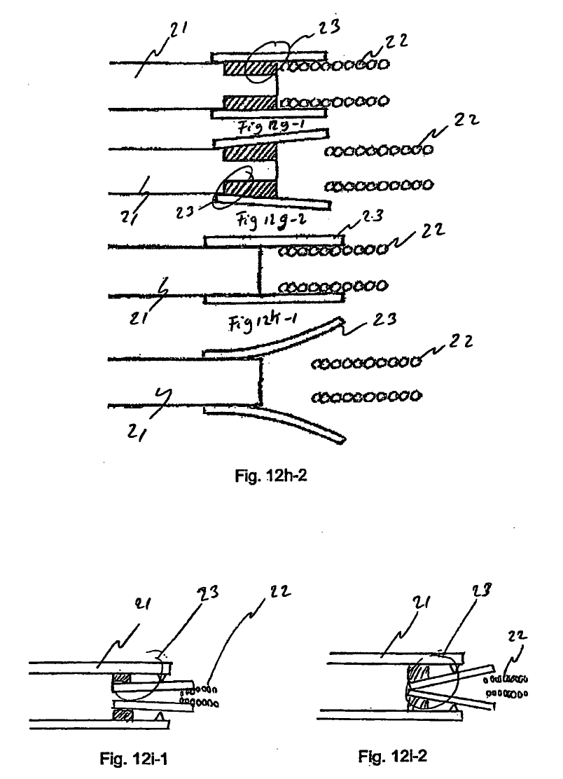

Yet another way to separate the two parts 21 and 22 is to design the EAP detachment actuator as a “wrapper” that tears the two part apart (FIG. 14a). This design is comparable to the plastic strip that is used to open plastic packages. Or when designed in the shape of FIG. 12i open/crack along the longitudinal direction like a banana. The EAP actuator may, for instance, be designed as a bending bilayer.

Yet, another way, as illustrated in FIG. 14b, may be use the EAP actuator to deform a portion of the second part 22 so that its diameter increases and thus can be released from the delivery device. This may be done by a volume changing EAP actuator.

Instead of deforming apportion of part 22, a portion of the second part 22 may be destroyed/ripped off/torn off as illustrated in FIG. 14c.

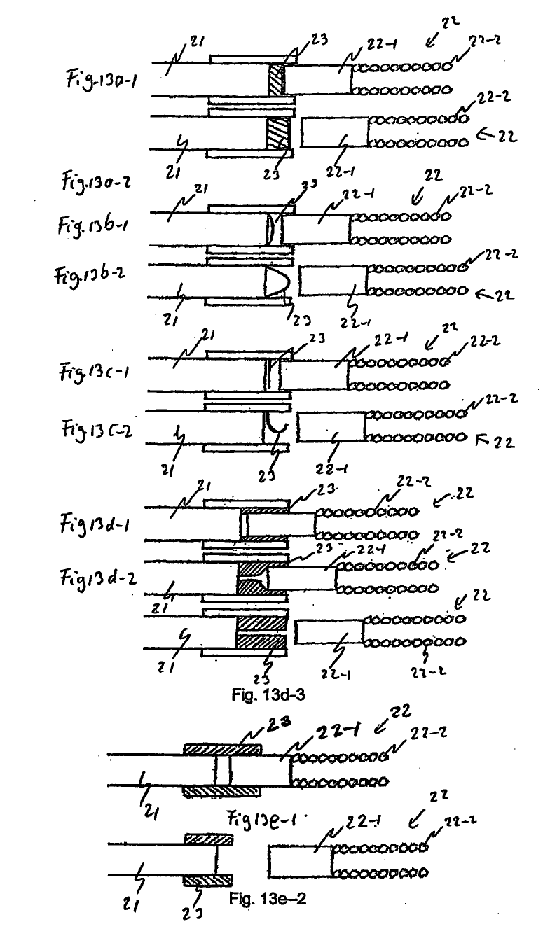

Yet another embodiment is shown in FIG. 15a. The detachment part 23 comprises an “electric glue” that releases upon electrical stimulation. Such electric glues are known from e.g. the following publicly available publications: Danielsson, C-O: Controlled Delamination Materials—Using Electrochemistry to Break Adhesive Joints in the Packaging Industry, Karlstad University, Department of Physics, Karlstad, Sweden; from Danielsson, C-O, Norberg, P. and Sandberg, L.: Controlled Delamination of Adhesives within Packaging and Distribution; and from WO2007/015675A1, the entire contents of which are incorporated herein by reference.

FIG. 15b is a variant of this embodiment. The EAP detachment part comprises two portions, one having a good adhesion, for instance the portion covering the first part 21, and the second portion has a changeable adhesion that is initially good, for instance the portion covering the second part 22. Upon activation of the EAP material, the adhesion between the part 22 and 23 deteriorates and part 22 is released from the delivery device. Such an embodiment may be achieved using the technology described in the above mentioned documents and in U.S. Pat. No. 6,103,399.

FIG. 15c illustrates an embodiment where the Young's modulus of the EAP material is changed. By electrically activating the EAP material, the material properties may be altered from having a high Young's modulus (stiff) to a low modulus (soft). In the soft state the part 22 can no longer be held by part 23 and part 22 is thus released.

As mentioned before the detachment part 23 may also be attached to the releasable part 22.

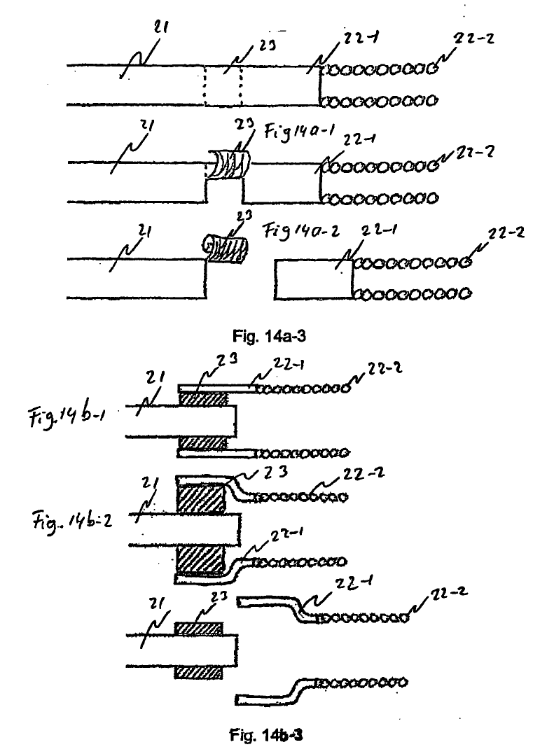

FIG. 16 illustrates two examples of such combinations. FIG. 16a shows an outwardly acting grip using a volume changing EAP actuator on the releasable part 22, i.e. a combination of FIG. 8b and FIG. 10a or of FIG. 11a and FIG. 8b).

Alternatively, FIG. 16b shows an inwardly acting grip using a volume changing EAP actuator on the releasable part 22, i.e. a combination of FIG. 8b and FIG. 10b or of FIG. 12a and FIG. 8b.

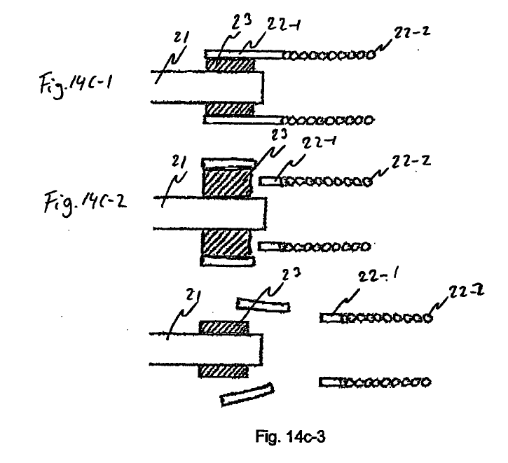

Yet another embodiment of the disclosure is shown in FIG. 17. The release device comprises four parts. In this case the detachment part 23 is positioned on a third part, that may be the catheter/protective tube 24 (13 in FIG. 6) and holds the releasable part 22. The releasable part 22 is pushed out using the push rod 21 (14) subsequent to detachment from part 23. The detachment part 23 may have any of the previously mentioned designs, as illustrated with respect to FIG. 7-15.

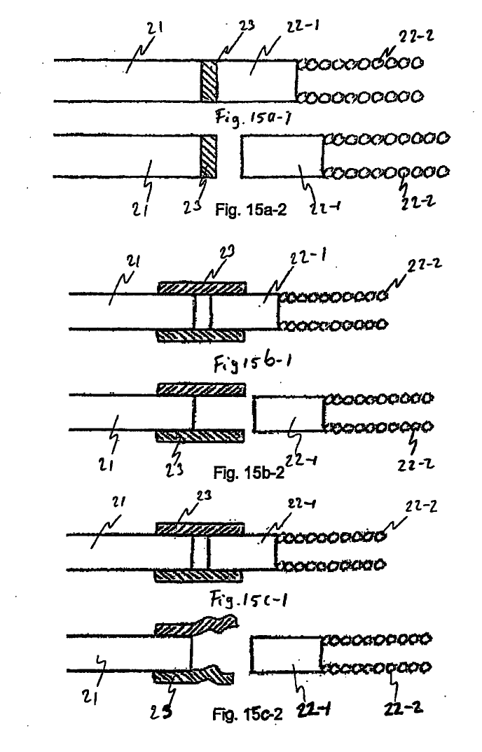

FIGS. 18 illustrate embodiments where one of the parts 21 or 22 comprises a means for accomplishing a good grip with the releasable part 23. One can compare this with a bushing.

In FIG. 18a both the delivery tool 21 and the releasable part 22 comprise a protrusion 26 respectively 25. The releasable part 23, here illustrated as being a volume changing actuator, keeps the releasable part 22 attachment by holding its protrusion 25 contained between the protrusion 26 and 23. Decreasing the diameter of the EAP part 23 (by activation) will enable the part 22 to be released.

Yet another example of bushing-like means is illustrated in FIG. 18b. Here the part 22 comprises an indent, notch, or groove that the part 21 to may engage. In this figure, tweezer-like hooks are illustrated. These hooks may be actuated by any of the previously mentioned EAP actuators, such as benders, volume expansions, see FIGS. 11 and 12. Likewise volume changing actuators may be used to engage such a notch.

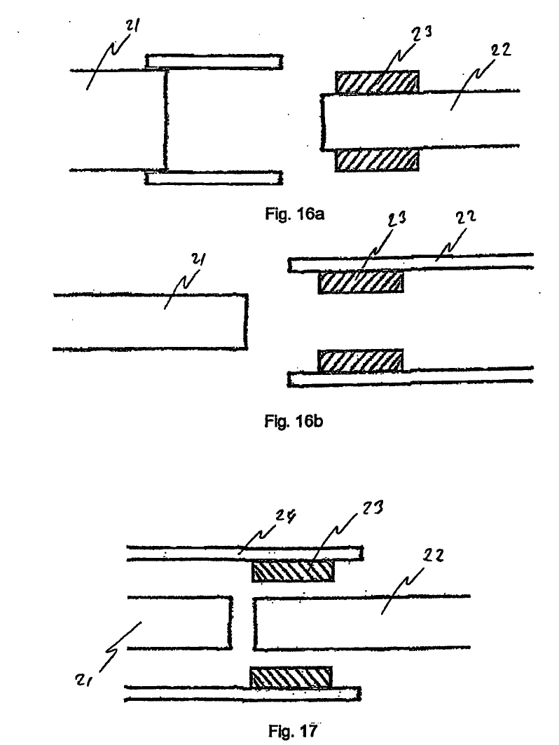

In the previous examples part 21 is illustrated as a delivery tool to be used under the surgical procedure only and removed from the body afterwards, whereas the part 22 is illustrated as an implant, or tool to be left into the body after the procedure. In an aspect illustrated in FIGS. 19a-19b, both 21 and 22 may be parts of an implant 30, that is possible to be divided on command into two or more separate parts 21 and 22 by activating the releasable part(s) 23, as is illustrated in FIG. 19. The mechanism for disconnecting the two parts 21, 22, may be any one of those previously illustrated.

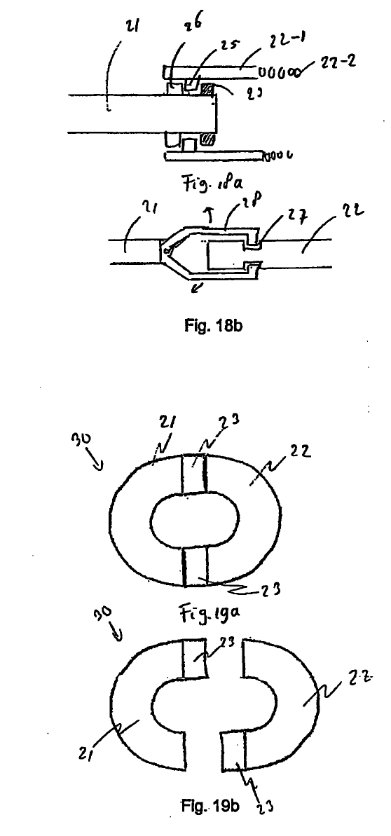

FIGS. 20a-20b illustrate embodiments where the parts 21 and 22 are interconnected by a fourth part 35, which may be a retaining part or linking part. This retaining part or linking part may be used in a single device as shown in FIG. 20a. Separating the parts 21 and 22 by activating the release part 23 increases the total length of the device. However, this may also be used as a part of the releasing mechanism 23, as shown in FIG. 20b, the second part 22 is released, and the retaining part or linking part is used to prevent the gripping members of e.g. FIG. 14c from totally separating from the first part 21, and possibly disappearing.

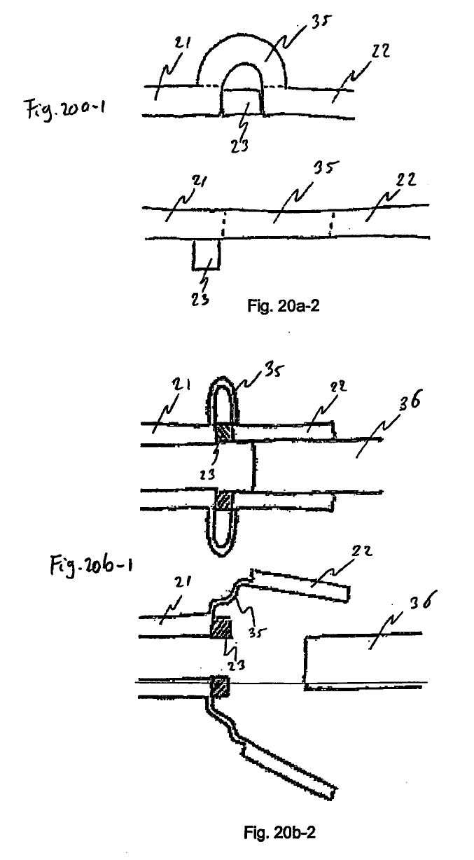

FIG. 21a illustrates an example of the structure illustrated in FIGS. 1c and 9 including a delivery device 40 comprising an array of tools/implants/parts, where multiple tools/implants/parts may be released from the same medical device.

The part 22a is attached to the delivery device 21 by detachment part 23a. In this example, the detachment parts 23a through 23c are designed as a bender that folds around the proximal portion of part 22a that is FIG. 12d. Part 22a further comprises a detachment part 23b to which the separately releasable part 22b is attached. Releasable part 22c is in its turn attached to part 22b by detachment part 23c. The whole assembly is mounted in the catheter/protection tube 13, which will prevent the benders from unfolding and thus releasing the detachment part 23c only.

Electrically activating the bender 23c, will open the bender so that the part 22c is set free, provided that the bender is not retained by the catheter/protection tube. The releasable parts/benders 23a-23c may be individually released by first activating detachment part 23c, then 23b and last 23a. Likewise the detachment parts/benders 23a-c may all be activated simultaneously. As the benders 23a and 23b are still contained inside the catheter 13, their motion is restrained and the parts 22a and 22b are still attached to the rod 21. Only bender 23c, that is pushed outside the catheter/tube 13, can move freely and expand, thus releasing the object 22c. Hereafter, the benders are deactivated/closed and the assembly is pushed further out of the catheter tube until bender 23b is outside. The benders are activated, only bender 23b can now move freely and releases part 22b. The procedure is repeated for as many objects as are needed for the procedure or until all the objects have been released. In this example the parts 22a through 22c are serially connected to each other. Likewise they may be all directly coupled to part 21.

Another way of creating individual actuation, while activating all EAP actuators simultaneously, is by controlling the electrolyte access. Taking FIG. 21b an example, the fit between parts 13 and 21, 22a, 22b, 23a, or 23b may be made tight, e.g. by providing a sleeve, so that the parts 23a and 23b have no access to the external, surrounding electrolyte. Only the part 23c that has been pushed outside the protective tube 13 has access to the electrolyte. When electrically activating, only the part that has access to the electrolyte, that is part 23c, can be operated, and thus only part 22c can be released.

The attachment parts 23a, 23b, 23c may be formed as any of the attachment parts described in the present disclosure.

FIGS. 22a through 22f illustrate different EAP actuator configurations or mechanisms that may be used in any of the previous embodiments, wherein electroactive polymer portions are indicated by 50, 50′, 50″ and passive portions are indicated by 51.

FIGS. 22a-1, 22a-2 and 22a-3 illustrate the concept of directly utilizing the volume change of the EAP material 50. One may either use the volume change in the perpendicular direction 50′ or longitudinal direction 50″.

Several layers of EAP might be stacked with alternating non-EAP layers, such as ion conducting layers or electrically conducting layers (FIGS. 22b-1, 22b-2). There might be several reasons for stacking such as to increase the total range of motion, increase force, or to increase the speed.

The EAP layer might be combined with at least a second layer, that may or may not be an EAP layer, see FIG. 22c-1, 22c-2. In this bilayer configuration, also addressed as unimorph, the volume change is transformed into a bending motion (benders).

The diameter of a circular part can be increased in several ways as exemplified in FIGS. 22d-1, 22d-2, 22d-3, 22d-4; 22d-5, 22d-6, 22d-7, 22d-8; and 22d-9, 22d-10,22d-11, 22d-12.

As illustrated in FIGS. 22d-1-22d-4, an annular shaped piece of EAP 50 will extend radially upon activation when utilizing the linear strain of the EAP volume change, either when formed as one single part or, as illustrated in FIG. 22d-11, comprising segmented EAP parts alternating with non-EAP parts.

As illustrated in FIGS. 22d-5-22d-12, the EAP may be combined with a structural unit such as a jointed or noded chain where the volume change is transformed into a diameter change. It is contemplated that even more complex structures, such as the Hoberman spheres, may be used. Not only the linear strain of the EAP material per se, may be used to generate a linear movement.

The bending movement may also be designed as a linear actuator, see FIG. 22e for several examples of such concepts: undulator or C-block configuration (FIGS. 22e-1-22e-2); bender/roll (FIGS. 22e-3-22e-4); buckling (FIGS. 22e-5-22e-6); benders/bilayers (FIGS. 22e-7-22e-8); and bellows (FIGS. 22e-9-22e-10).

The bulk volume change may also be used in connection with a spinal structure to generate a bending motion as exemplified in FIG. 22f: V-grooves (FIG. 22f-1-22f-2), spine (FIG. 22f-3-22f-4); coil (FIG. 22f-5-22f-6). Also, a pulley kind of construction (FIG. 22f-7-22f-8) where the linear strain pulls the actuator into a bending shape may be used.

As yet another option, a volume changing actuator can be used to create a tweezer-like motion. FIG. 22f-9-22f-10 illustrates a single tweezer member, similar to that of FIGS. 12i-1 and 12i-2.

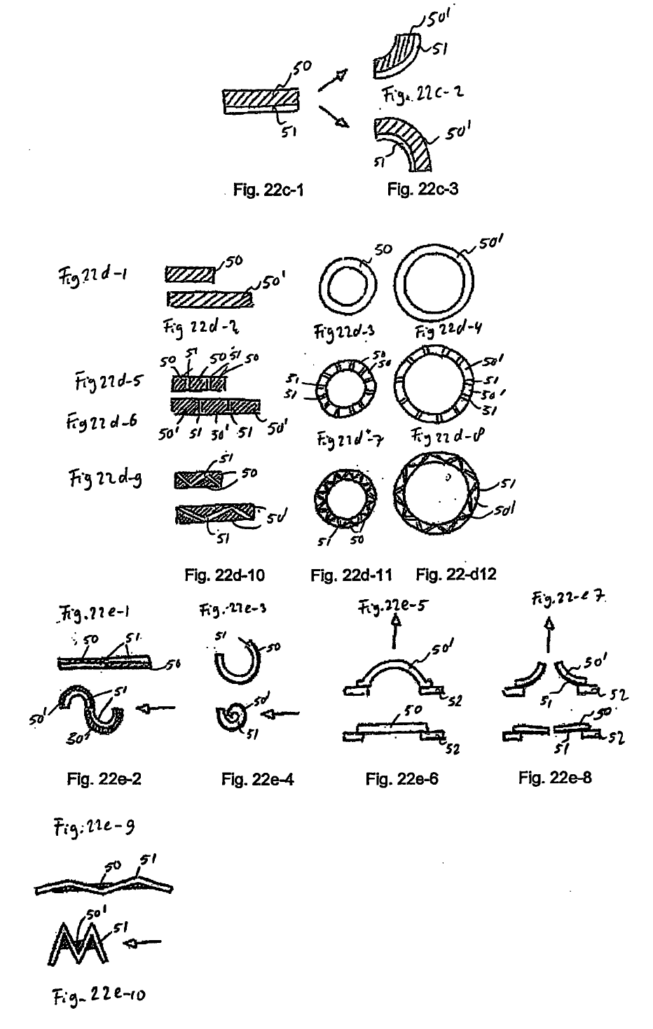

In FIG. 23a, there is illustrated an insertion part 21, which may be a carrier (needle etc.) or a catheter, having a polymer microactuator 23 acting outwardly to directly engage a tool, which here is in the form of an aneurysm coil.

In FIG. 23b, there is illustrated an insertion part 21, which may be a carrier (needle etc.) or a catheter, and a tool, which here is in the form of an aneurysm coil, having a polymer microactuator 23 acting inwardly to directly engage the insertion part 21.

In FIG. 23c, there is illustrated an insertion part 21, which may be a carrier (needle etc.) or a catheter having a polymer microactuator 23 acting outwardly to engage an attachment portion 22-1 of a tool 22, which here is in the form of an aneurysm coil.

In FIG. 23d, there is illustrated an insertion part 21, which may be a carrier (needle etc.) or a catheter, and a tool, which here is in the form of an aneurysm coil, having an attachment portion 22-1 with a polymer microactuator 23 acting inwardly to directly engage the insertion part 21.

In FIG. 23e, there is illustrated an insertion part 21, which may be a carrier (needle etc.) or a catheter, and a tool, which here is in the form of an aneurysm coil, having a polymer microactuator 23 arranged directly on the coil and acting outwardly to engage an enclosing attachment portion of the insertion part.

In FIG. 23f, there is illustrated an insertion part 21, which may be a carrier (needle etc.) or a catheter having a tool-enclosing attachment portion with a polymer microactuator 23 acting inwardly to directly engage a tool 22, which here is in the form of an aneurysm coil.

In FIG. 23g, there is illustrated an insertion part 21, which may be a carrier (needle etc.) or a catheter, and a tool, which here is in the form of an aneurysm coil, having a polymer microactuator 23 arranged on an attachment portion 22-1 of the tool, and acting outwardly to engage an enclosing attachment portion of the insertion part.

In FIG. 23h, there is illustrated an insertion part 21, which may be a carrier (needle etc.) or a catheter having a tool-enclosing attachment portion with a polymer microactuator 23 acting inwardly to engage an attachment portion 22-1 of a tool 22, which here is in the form of an aneurysm coil.

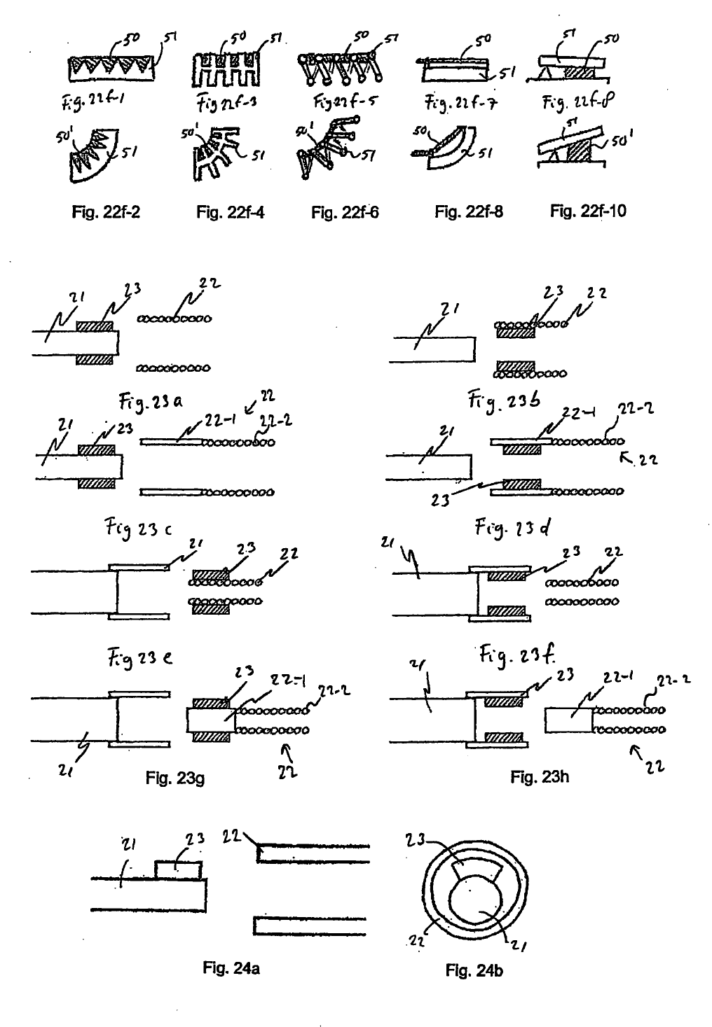

In FIGS. 24a-24b, there is illustrated an embodiment, wherein an outwardly acting actuator 23 is arranged with a limited circumferential extent. The actuator 23 may be according to any of the principles disclosed in this disclosure. Corresponding embodiments may be provided with inwardly acting actuators, analogously with what was disclosed in FIGS. 12a, 12b, 12c, 13d, 16a, 16b and 17. Particular embodiments of the present disclosure are detailed in the numbered paragraphs set forth below. While these paragraphs at present do not constitute patent claims, the right to claim the subject matter described below is retained.

1. A surgical device, comprising:

an insertion device, adapted for insertion into a body,

a tool, which is to be inserted and left in the body, and

a polymer microactuator, arranged to releasably retain the tool in or on the insertion device.

2. The device as described in paragraph 1, wherein the tool is releasably retainable inside the insertion device.

3. The device as described in paragraph 2, wherein the insertion device comprises a catheter or a cannula.

4. The device as paragraphed in any one of the preceding paragraphs, wherein a carrier device is insertable into the insertion device.

5. The device as described in paragraph 4, wherein the tool is releasably retainable on the carrier device.

6. The device as described in paragraph 5, wherein the carrier device comprises a needle.

7. The device as paragraphed in any one of the preceding paragraphs, wherein the polymer microactuator is arranged in or on the insertion device.

8. The device as described in paragraph 7, wherein the polymer microactuator comprises a portion, which extends axially beyond a distal part of an insertion device body.

9. The device as paragraphed in any one of the preceding paragraphs, wherein the polymer microactuator is arranged in or on the tool.

10. The device as described in paragraph 9, wherein the polymer microactuator comprises a portion, which extends axially beyond a distal part of a tool body.

11. The device as described in any one of the preceding paragraphs, wherein the tool is axially displaceable in or on the insertion device.

12. The device as described in any one of the preceding paragraphs, wherein the polymer microactuator is arranged in or on a third part.

13. The device as described in paragraph 12, wherein the polymer microactuator comprises a portion, which extends axially beyond a distal part of a third part body.

14. The device as described in paragraph 12 or 13, wherein the tool and/or the third part is axially displaceable in or on the insertion device.

15. The device as described in any one of the preceding paragraphs, wherein the tool at least partially encloses a portion of the insertion device.

16. The device as described in paragraph 15, wherein the polymer microactuator is arranged on the insertion device.

17. The device as described in paragraph 16, wherein the polymer microactuator is arranged on an outer surface of the insertion device and outwardly expandable to engage an inner surface of the tool.

18. The device as described in any one of paragraphs 1-14, wherein the insertion device at least partially encloses a portion of the tool.

19. The device as described in paragraph 18, wherein the polymer microactuator is arranged on the tool.

20. The device as described in paragraph 19, wherein the polymer microactuator is arranged on an inner surface of the insertion device and inwardly expandable to engage an outer surface of the tool.

21. The device as described in any one of the preceding paragraphs, wherein the tool and the insertion device are connectable and disconnectable by a substantially axial mutual displacement.

22. The device as described in any one of paragraphs 1-20, wherein the tool and the insertion device are connectable and disconnectable by a substantially radial mutual displacement.

23. The device as described in paragraph 22, wherein one of the insertion device and the tool is radially insertable into a recess in the other one of the insertion device and the tool.

24. The device as described in paragraph 22 or 23, wherein the insertion device and the tool are interconnectable by a combination of radial and axial movements.

25. The device as described in any one of the preceding paragraphs, wherein the insertion device and the tool are interconnectable in a position where longitudinal axes of the insertion device and the tool are angled relative each other.

26. The device as described in any one of paragraphs 1-24, wherein the insertion device and the tool are interconnectable in a position where longitudinal axes of the insertion device and the tool are substantially parallel.

27. The device as paragraphed any one of the preceding paragraphs, wherein the polymer microactuator comprises at least two separate polymer microactuator portions.

28. The device as described in paragraph 27, wherein the polymer microactuator portions are axially juxtaposed or spaced.

29. The device as described in paragraph 27 or 28, wherein the polymer microactuator portions are angularly juxtaposed or spaced.

30. The device as described in any one of paragraphs 27-29, wherein one of the polymer microactuator portions is arranged on one of the insertion device, the tool and a third part and another one of the polymer microactuator portions is arranged on another one of the insertion device, the tool and the third part.

31. The device as described in any one of the preceding paragraphs, wherein the polymer microactuator comprises a polymer material block, which is expandable and/or contractable in a radial direction.

32. The device as described in paragraph 31, wherein the material block is substantially annular.

33. The device as paragraphed in any one of the preceding paragraphs, wherein the polymer microactuator comprises a bellows or undulated portion.

34. The device as described in paragraph 33, wherein the bellows or undulated portion is expandable and/or contractable in a radial direction.

35. The device as paragraphed in any one of the preceding paragraphs, wherein the polymer microactuator comprises a buckling portion.

36. The device as described in paragraph 35, wherein the polymer microactuator comprises at least two buckling portions.

37. The device as paragraphed in any one of the preceding paragraphs, wherein the polymer microactuator comprises an inflatable portion.

38. The device as paragraphed in any one of the preceding paragraphs, wherein the polymer microactuator comprises a bending portion.

39. The device as described in paragraph 38, wherein the bending portion is bendable about a transversal direction of the surgical device.

40. The device as described in paragraph 38, wherein the bending portion is arranged as an at least partial collar.

41. The device as described in paragraph 38, wherein the bending portion is bendable about an axial direction of the surgical device.

42. The device as described in paragraph 41, wherein the polymer microactuator comprises at least two separate bending portions.

43. The device as described in paragraph 42, wherein the bending portions extend from one of the insertion device, the tool and the third part.

44. The device as described in paragraph 43, wherein the bending portions extend towards the same rotational direction.

45. The device as described in paragraph 43, wherein the bending portions extend towards different rotational directions.

46. The device as described in any one of paragraphs 38-45, wherein the polymer microactuator is arranged to bend a portion of the insertion device.

47. The device as described in any one of the preceding paragraphs, wherein the polymer microactuator comprises a tweezer portion.

48. The device as described in paragraph 47, wherein the tweezer portion comprises gripping member, arranged on one of the insertion device, the tool and the third part for interaction with the other one of the insertion device, the tool and the third part.

49. The device as described in paragraph 48, wherein the gripping member is substantially rigid.

50. The device as described in paragraph 48, wherein the gripping member is bendable.

51 The device as described in paragraph 50, wherein the actuator is integrated with the gripping member.

52. The device as paragraphed in any one of the preceding paragraphs, wherein the polymer microactuator comprises a first portion, which is attached to one of the insertion device, the tool and the third part, and a second portion, arranged to engage an other one of the insertion device, the tool and the third part.

53. The device as described in paragraph 52, wherein the second portion is radially expandable/contractable.

54. The device as described in paragraph 52, wherein the second portion is axially expandable/contractable.

55. The device as described in any one of the preceding paragraphs, wherein the polymer microactuator is arranged on one of the insertion device, the tool and the third part, and is arranged to squeeze out the other one of the insertion device, the tool and the third part.

56. The device as described in any one of the preceding paragraphs, wherein one of the insertion device, the tool and the third part, if any, comprises a permanently deformable or breakable portion, which is deformed or broken, respectively, through actuation of the polymer microactuator.

57. The device as described in paragraph 56, wherein said one of the insertion device, the tool and the third part comprises a severance mark.

58. The device as described in paragraph 57, wherein the severance mark extends along a circumference of said one of the insertion device, the tool and the third part.

59. The device as described in paragraph 58, wherein the severance mark extends along a length of said one of the insertion device, the tool and the third part.

60. The device as described in paragraph 57, wherein one of the insertion device, the tool and the third part comprises a sleeve.

61 The device as described in paragraph 58, wherein the sleeve is permanently deformable through actuation of the polymer microactuator.

62. The device as described in paragraph 57, wherein the sleeve is breakable through actuation of the polymer microactuator.

63. The device as described in any one of paragraphs 56-62, further comprising a retaining portion, arranged to retain said deformable or breakable portion subsequent to said actuation of the polymer microactuator.

64. The device as described in any one of the preceding paragraphs, wherein the polymer microactuator comprises a variable adhesion portion having electrically controllable adhesion properties.

65. The device as described in paragraph 64, wherein the variable adhesion portion comprises electrically controllable glue.

66. The device as described in paragraph 64, wherein the variable adhesion portion comprises a laminated structure, which upon actuation is arranged to delaminate upon actuation of the polymer microactuator.

67. The device as described in any one of the preceding paragraphs, wherein the polymer microactuator comprises a variable elasticity portion having electrically controllable modulus of elasticity.

68. The device as described in paragraph 67, wherein the variable elasticity portion, upon actuation, is arranged to release the tool as a direct consequence of an increase or decrease of the modulus of elasticity.

69. The device as described in any one of the preceding paragraphs, wherein the polymer microactuator is arranged as a variable locking element one of the insertion device, the tool and the third part, for positive interlocking with a corresponding part on another one of the insertion device, the tool and the third part.

70. The device as described in any one of the preceding paragraphs, wherein the polymer microactuator is arranged on one of the insertion device, the tool and the third part, to control a gripping device, which is arranged to interact with another one of the insertion device, the tool and the third part.

71. The device as described in paragraph 70, wherein the gripping device is arranged for interaction through positive interlocking with a corresponding part on said another one of the insertion device, the tool and the third part.

72. The device as described in paragraph 69 or 71, wherein the corresponding part is a protrusion, a groove or a variable locking element.

73. The device as described in any one of the preceding paragraphs, wherein an array comprising at least two tools are arranged in or on the insertion device, and wherein an outermost tool is releasable through actuation of the polymer microactuator.

74. The device as described in paragraph 73, wherein the insertion device is arranged to prevent release of all but the outermost tool.

75. The device as described in paragraph 74, wherein the insertion device is arranged to mechanically prevent release of all but the outermost tool.

76. The device as described in paragraph 74, wherein the insertion device is arranged to prevent release of all but the outermost tool by preventing sufficient electrolyte access to at least one of the tools.

77. The device as described in paragraph 76, wherein the insertion device comprises a seal, which is operable against at least one of the tools.

78. The device as described in any one of the preceding paragraphs, wherein the tool comprises an aneurysm coil.

79. The device as described in any one of the preceding paragraphs, wherein the tool comprises an active tool part and an attachment part for interaction with the insertion device or third part.

80. The device as described in any one of the preceding paragraphs, wherein the insertion device comprises an elongate tubular device, to which the tool is releasably attachable.

81. The device as described in any one of the preceding paragraphs, wherein the insertion device comprises a solid elongate device, to which the tool is releasably attachable.

82. A body-implantable device, comprising:

first and second passive device portions, and

an attachment device for releasably attaching said first and second device portions to each other,

wherein said attachment device comprises a polymer microactuator.

83. The device as described in paragraph 82, wherein said said first and second device portions are formed in one piece.

84. The device as described in paragraph 82 or 83, further comprising a retaining portion, arranged to retain said first and second portions subsequent to said actuation of the polymer microactuator.

85. The device as described in paragraph 84, wherein the retaining portion is arranged to, subsequent to said actuation, retain said first and second portions at a larger distance from each other than prior to said actuation.

86. The device as claimed in any one of paragraphs 82-85, wherein said first and second parts are arranged to form an encircling device, the circumference of which being larger subsequent to said actuation, than prior to said actuation.

It is noted that, while a few embodiments are illustrated in the present disclosure, a large number of variations and combinations are possible. For example, it is contemplated that any combination of the position of the polymer microactuator (FIGS. 8a-8d), gripping direction (FIGS. 10a-10d) and actuator type (FIGS. 11a-18b, 22a-22f) may be used to provide a surgical device.

The devices illustrated herein may be circular in cross section, where the detachment portions may be devised as rings/annular shaped or the devices may have a rectangular (or other shape) cross section and the detachment portions formed as two opposing parts, one on the top and one on the bottom of the device.

In this disclosure the release of an object is described. As one skilled in the art realize the many of the methods may be used to grab and capture an object, such as a blood cloth, tissue (part), a medical device part or implant that is to be retrieved from the body after completing an intervention or treatment etc.

While the above description contains many specifications, these should not be construed as limitations on the scope of the invention, but as exemplifications of the presently preferred embodiment thereof. Many other ramifications and variations are possible within the scope of the appended claims.

1. A surgical device, comprising:
an insertion device, adapted for insertion into a body,
a tool, which is to be inserted and left in the body, and
a polymer microactuator, arranged to releasably retain the tool in or on the insertion device.

2. The device as claimed in claim 1, wherein the tool is releasably retainable inside the insertion device.

3. The device as claimed in claim 2, wherein the insertion device comprises a catheter or a cannula.

4. The device as claimed in claim 1, wherein a carrier device is insertable into the insertion device.

5. The device as claimed in claim 4, wherein the tool is releasably retainable on the carrier device.

6. The device as claimed in claim 5, wherein the carrier device comprises a needle.

8. The device as claimed in claim 1, wherein the polymer microactuator comprises an electroactive polymer.

9. The device as claimed in claim 8, wherein the electroactive polymer changes volume upon actuation.

10. The device as claimed in claim 1, wherein the polymer microactuator is arranged in or on the insertion device.

11. The device as claimed in claim 1, wherein the polymer microactuator is arranged in or on the tool.

12. The device as claimed in claim 1, wherein the tool at least partially encloses a portion of the insertion device.

13. The device as claimed in claim 1, wherein the insertion device at least partially encloses a portion of the tool.

14. A method for operating a surgical device, comprising:
providing an insertion device, adapted for insertion into a body,
providing a tool, which is to be inserted and left in the body, and which is releasably attached to the insertion device, and
actuating a polymer microactuator, so as to release the tool from the insertion device.

15. A body-implantable device, comprising:
first and second device passive portions, and
an attachment device for releasably attaching said first and second device portions to each other,
wherein said attachment device comprises a polymer microactuator.

16. The device as claimed in claim 15, wherein said first and second device portions are formed in one piece.

17. The device as claimed in claim 15, further comprising a retaining portion, arranged to retain said first and second portions subsequent to said actuation of the polymer microactuator.

18. The device as claimed in claim 17, wherein the retaining portion is arranged to, subsequent to said actuation, retain said first and second portions at a larger distance from each other than prior to said actuation.

19. The device as claimed in claim 15, wherein said first and second parts are arranged to form an encircling device, the circumference of which being larger subsequent to said actuation, than prior to said actuation.
